(19) United States
(12) Patent Application Publication
(10) Pub. No.: US 2023/0172882 A1
(43) Pub. Date: Jun. 8, 2023

(54) Method For Treating Pancreatic Cancer

(71) Applicant: Lantern Pharma Inc., Dallas, TX (US)

(72) Inventors: Aditya Kulkarni, Dallas, TX (US); Kishor Bhatia, Dallas, TX (US)

(21) Appl. No.: 17/998,117

(22) PCT Filed: May 10, 2021
(86) PCT No.: PCT/US2021/031606

§ 371 Date: Nov. 7, 2022

Related U.S. Application Data
(60) Provisional application No. 63/022,242, filed on May 8, 2020.

(51) Int. Cl. A61K 31/17; A61P 1/18; A61K 31/495; A61K 39/395; A61K 31/436; A61K 31/175; A61K 31/166; A61K 31/475; A61K 31/4745; A61K 33/243; A61K 31/282; A61K 31/519; A61K 31/357; A61K 31/7064; A61K 38/12; A61K 31/675; A61P 35/00

(52) U.S. Cl. CPC A61K 31/17; A61P 1/18; A61K 31/495; A61K 39/3955; A61K 31/436; A61K 31/175; A61K 31/166; A61K 31/475; A61K 31/4745; A61K 33/243; A61K 31/282; A61K 31/519; A61K 31/357; A61K 31/7064; A61K 38/12; A61K 31/675; A61P 35/00

(57) ABSTRACT

Methods for treating pancreatic cancer by administration hydroxyureamethyl acylfulvene. Some embodiments relate to treatment of pancreatic cancer by administration of hydroxyureamethyl acylfulvene.

### TECHNICAL FIELD

This application relates to the field of chemistry and oncology. More particularly, this application relates to methods for treating pancreatic cancer using hydroxyureamethyl acylfulvene.

### BACKGROUND

Pancreatic cancer has one of the highest mortality rates among all cancers and is expected to cause over 60,000 deaths in the United States. For all stages of pancreatic cancer combined, the 1 to 5-year relative survival rates are low, respectively; this high mortality rate from pancreatic cancer is, at least in part, due to the high incidence of metastatic disease at the time of diagnosis. As a result, treatment options for pancreatic cancer are very limited.

Further, pancreatic cancer is hard to catch early because symptoms do not appear right away. When there are symptoms, such symptoms are often vague or hard to notice. Because the pancreas is hidden behind other organs, health care providers cannot see or feel the tumors during routine exams. Doctors use a physical exam, blood tests, imaging tests, and a biopsy to diagnose it. Because pancreatic cancer is often found late and it spreads quickly, pancreatic cancer can be hard to treat.

Accordingly, there is a need for treatments for primary and metastatic pancreatic cancers.

### BRIEF DESCRIPTION OF THE FIGURES

FIG. 1 shows the cytotoxicity of hydroxyureamethyl acylfulvene or LP-184 in terms of IC50 values in a first study of 4 pancreatic cancer cell lines.

FIG. 2 shows the cytotoxicity of hydroxyureamethyl acylfulvene or LP-184 in terms of IC50 in values in a second study of 6 pancreatic cancer cell lines.

FIG. 3 shows IC50 values from tissue from prostate cancer tissue models that were treated ex vivo with hydroxyureamethyl acylfulvene or LP-184.

FIG. 4 shows tumor volume growth in xenograft mouse models over 60 days in mice treated with hydroxyureamethyl acylfulvene or LP-184.

### SUMMARY

One aspect of this application includes a method of treating pancreatic cancer, in a subject in need thereof, the method includes administering to the subject a therapeutically effective amount of hydroxyureamethyl acylfulvene. The pancreatic cancer can be endocrine or exocrine. The pancreatic cancer can pancreatic adenocarcinoma. In one embodiment, the subject or subject cancer can express a high level of PTGR1. The pancreatic cancer can be Stage I, II, III, or IV

Another aspect of this application includes the treatment of patients suffering from one or more of pancreatic adenocarcinoma, non-resectable pancreatic cancer, locally advanced pancreatic cancer, borderline resectable pancreatic cancer, locally advanced pancreatic ductal adenocarcinoma, borderline resectable pancreatic ductal adenocarcinoma, metastatic pancreatic cancer, chemotherapy-resistant pancreatic cancer, pancreatic ductal adenocarcinoma, squamous pancreatic cancer, pancreatic progenitor, immunogenic pancreatic cancer, aberrantly differentiated endocrine exocrine (ADEX) tumors, an exocrine pancreatic cancer, pancreatic intraepithelial neoplasia, intraductal papillary mucinous neoplasms, mucmous cystic neoplasms, mucmous pancreas cancer, adenosquamous carcinoma, signet ring cell carcinoma, hepatoid carcinoma, colloid carcinoma, undifferentiated carcinoma, undifferentiated carcinomas with osteoclast-like giant cells, a pancreatic cystic neoplasm, an islet cell tumor, a pancreas endocrine tumor, or a pancreatic neuroendocrine tumor.

Another aspect includes a treatment also including the use of one or more additional therapeutic agents, e.g., selected from the group consisting of temozolomide, bevacizumab, everolimus, carmustine, lomustine, procarbazine, vincristine, irinotecan, cisplatin, carboplatin, methotrexate, etoposide, vinblastine, bleomycin, actinomycin, cyclophosphamide, and ifosfamide.

Another aspect includes the use of one or more additional therapeutic agents, e.g., selected from the group consisting of cisplatin, paclitaxel, and combination thereof.

Another aspect includes a treatment also including subjecting the subject to radiation therapy. The subject or patient may have undergone surgery, radiation therapy, and chemotherapy.

Another aspect includes a method of treating pancreatic cancer a subject including (a) obtaining or having obtained an expression level in a sample from a subject for a plurality of targets, wherein the plurality of targets comprises includes PTRG1; (b) determining that the subject is sensitive to a treatment with a hydroxyureamethyl acylfulvene; and (c) administering a cancer treatment including a hydroxyureamethyl acylfulvene. The patient may have undergone or may be undergoing radiation therapy.

### Definitions

Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “antibody” as used herein includes intact molecules as well as molecules comprising or consisting of fragments thereof, such as, for example, Fab, F(ab′)2, Fv and scFv, as well as engineered variants including diabodies, triabodies, mini-bodies and single-domain antibodies which are capable of binding an epitopic determinant. Thus, antibodies may exist as intact immunoglobulins, or as modifications in a variety of forms.

The term “biomarker” refers to any molecule, such as a gene, gene transcript (for example mRNA), peptide or protein or fragment thereof produced by a subject which is useful in differentiating subjects to predict the responsiveness of patients to treatments including hydroxyureamethyl-acylfulvene or its analogs. A biomarker that is differentially present (i.e., increased or decreased) in a biological sample from a subject or a group of subjects has a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using either Welch's T-test or Wilcoxon's rank-sum Test).

The term “pancreatic cancer” refers to a neoplasm that originates in the pancreas. Pancreatic cancer includes exocrine and endocrine cancers. Most pancreatic cancers are exocrine tumors. Pancreatic endocrine tumors are also called islet cell tumors. Pancreatic cancers that can be treated with methods described herein include, but are not limited to, exocrine pancreatic cancers and endocrine pancreatic cancers. Exocrine pancreatic cancers include, but are not limited to, adenocarcinomas, acinar cell carcinomas, adenosquamous carcinomas, colloid carcinomas, undifferentiated carcinomas with osteoclast-like giant cells, hepatoid carcinomas, intraductal papillary-mucinous neoplasms, mucinous cystic neoplasms, pancreatoblastomas, serous cystadenomas, signet ring cell carcinomas, solid and pseuodpapillary tumors, pancreatic ductal carcinomas, and undifferentiated carcinomas. In some embodiments, the exocrine pancreatic cancer is pancreatic ductal carcinoma. Endocrine pancreatic cancers include, but are not limited to, insulinomas and glucagonomas.

The terms “patient,” “subject,” “individual,” and “host” refer to either a human or a non-human animal suffering from or suspected of suffering from a disease or disorder associated with aberrant biological or cell growth activity.

The term “preventing” when used in relation to a condition or disease such as cancer, refers to a reduction in the frequency of, or delay in the onset of, symptoms of the condition or disease. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

The term “a subject in need thereof” refers to a subject whom has been diagnosed as suffering from the specific condition intended to be treated, e.g., pancreatic cancer or a specific type of pancreatic cancer.

The terms “expression level” and “level of expression,” as used herein, refer to the amount of a gene product in a cell, tissue, biological sample, organism, or patient, e.g., amounts of DNA, RNA (e.g. messenger RNA (mRNA)), or proteins corresponding to a given gene.

The term “pharmaceutically acceptable” means that, which is useful in preparing a pharmaceutical composition that is generally safe, non-toxic, and neither biologically nor otherwise undesirable and includes that which is acceptable for veterinary as well as human pharmaceutical use.

The term “healthy individual” shall be taken to mean an individual who is known not to suffer from cancer (e.g., cancer), such knowledge being derived from clinical data on the individual, including, but not limited to, a different diagnostic assay to that described herein.

The term “pharmaceutically acceptable salt” refers to a salt which is acceptable for administration to a patient, such as a mammal (e.g., salts having acceptable mammalian safety for a given dosage regime). Such salts can be derived from pharmaceutically acceptable inorganic or organic bases and from pharmaceutically-acceptable inorganic or organic acids, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Salts derived from pharmaceutically acceptable inorganic bases include aluminum, ammonium, calcium, copper, ferric, ferrous, lithium, magnesium, manganic, manganous, potassium, sodium, zinc and the like. Salts derived from pharmaceutically acceptable organic bases include salts of primary, secondary, tertiary and quaternary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, N,N′-dibenzylethylenediamine, chloroprocaine, choline, diethanolamine, meglumine (N-methyl-glucamine) and the like. When compounds of the present disclosure contain relatively basic functionalities, acid addition salts can be obtained by contacting the neutral form of such compounds with a sufficient amount of the desired acid, either neat or in a suitable inert solvent. Salts derived from pharmaceutically acceptable acids include acetic, trifluoroacetic, propionic, ascorbic, benzenesulfonic, benzoic, camphosulfonic, citric, ethanesulfonic, fumaric, glycolic, gluconic, glucoronic, glutamic, hippuric, hydrobromic, hydrochloric, isethionic, lactic, lactobionic, maleic, malic, mandelic, methanesulfonic, mucic, naphthalenesulfonic, nicotinic, nitric, pamoic, pantothenic, phosphoric, succinic, sulfuric, hydroiodic, carbonic, tartaric, p-toluenesulfonic, pyruvic, aspartic, benzoic, anthranilic, mesylic, salicylic, p-hydroxybenzoic, phenylacetic, embonic (pamoic), ethanesulfonic, benzenesulfonic, 2-hydroxyethanesulfonic, sulfanilic, stearic, cyclohexylaminosulfonic, algenic, hydroxybutyric, galactaric and galacturonic acid and the like.

The term “effective amount” refers to an amount of a compound or composition sufficient to treat a specified disorder, condition or disease such as ameliorate, palliate, lessen, and/or delay one or more of its symptoms. In reference to pancreatic cancer, an effective amount comprises an amount sufficient to cause a tumor to shrink and/or to decrease the growth rate of the tumor (such as to suppress tumor growth) or to prevent or delay other unwanted cell proliferation in pancreatic cancer. In some embodiments, an effective amount is an amount sufficient to delay development of pancreatic cancer. In some embodiments, an effective amount is an amount sufficient to prevent or delay recurrence. An effective amount can be administered in one or more administrations. In the case of pancreatic cancer, the effective amount of the drug or composition may: (i) reduce the number of pancreatic cancer cells; (ii) reduce tumor size; (iii) inhibit, retard, slow to some extent and preferably stop pancreatic cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; (vii) relieve to some extent one or more of the symptoms associated with pancreatic cancer; and/or (viii) disrupt (such as destroy) pancreatic cancer stroma.

The phrase “therapeutically effective amount” means an amount of an active agent that (i) treats or prevents pancreatic cancer, (ii) attenuates, ameliorates, or eliminates one or more symptoms of pancreatic cancer, or (iii) prevents or delays the onset of one or more symptoms of pancreatic cancer. The therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The term “therapeutic effect” refers to a beneficial local or systemic effect in animals, particularly mammals, and more particularly humans, caused by administration of a compound or composition of the invention. The phrase “therapeutically-effective amount” means that the amount of a compound or composition of the invention that is effective to treat a disease or condition caused by aberrant biological activity at a reasonable benefit/risk ratio. In some embodiments, the therapeutically effective amount of hydroxyureamethyl-acylfulvene or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day.

A “reference level” means a level of the compound of the present invention or additional biomarker(s) that is indicative of a particular disease state, phenotype, or lack thereof, as well as combinations of disease states, phenotypes, or lack thereof.

A “reference sample” refers to a sample containing reference level of a biomarker. For example, a reference sample can be obtained from a subject that does not have a particular disease, disease state or phenotype, such as cancer or acute injury.

The therapeutically effective amount of such substance will vary depending upon the subject and disease condition being treated, the weight and age of the subject, the severity of the disease condition, the manner of administration and the like, which can readily be determined by one of skill in the art.

### DETAILED DESCRIPTION

HydroxyUreaMethyl Acylfulvene (currently, termed as LP-184 by Lantern Pharma, Inc.) is a semisynthetic or synthetic antitumor agent derived from the mushroom toxin illudin S. The structure of hydroxyureamethyl acylfulvene is shown below:

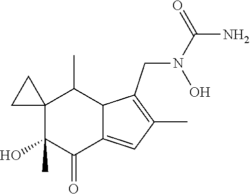 [SMILES: CC1=C(CN(O)C(N)=O)C2C(=C1)C(=O)[C@](C)(O)C1(CC1)C2C]

Specific embodiments provide methods for the treatment of pancreatic cancer (e.g., metastatic pancreatic cancer or locally advanced unresectable pancreatic cancer) in a subject (e.g., human) using hydroxyureamethyl acylfulvene or salt thereof. Specific embodiments relate to methods of treating pancreatic cancer, the methods including the administration of an effective amount of hydroxyureamethyl acylfulvene or salt thereof to a subject in need thereof. In one example, hydroxyureamethyl acylfulvene can be administered as a monotherapy. The pancreatic cancer can be at early stage or at late stage and may have also metastasized. The combinations described herein can be used to treat cancers at any stage, including those cancers that have metastasized.

One embodiment includes co-administering hydroxyureamethyl acylfulvene and an additional therapeutic agent in separate composition or the same composition. Thus, some embodiments include a first pharmaceutical composition comprising: (a) a safe and therapeutically effective amount of hydroxyureamethyl acylfulvene or pharmaceutically acceptable salts thereof and (b) a pharmaceutically acceptable carrier, diluent, excipient or combination thereof; and a second pharmaceutical composition comprising: (a) a safe and therapeutically effective amount of an additional therapeutic agent and (b) a pharmaceutically acceptable carrier, diluent, excipient or combination thereof. Some embodiments include a pharmaceutical composition comprising: (a) a safe and therapeutically effective amount of an additional therapeutic agent; and (b) a pharmaceutically acceptable carrier, diluent, excipient or combination thereof. In some embodiments, the method described herein can further include subjecting the subject to a radiation therapy. In some embodiments, at least about 10% (including for example at least about any of 20%, 30%, 40%, 60%, 70%, 80%, 90%, 91%, 92%, 93%, 94%, 95%, 96%, 97%, 98%, 99%, or 100%) cell proliferation is inhibited.

In some embodiments, the pancreatic cancer to be treated is stage 0, stage I, stage II, stage III, or stage IV. In some embodiments, the pancreatic cancer to be treated is stage 0, stage IA, stage IB, stage HA, stage IIB, stage III, or stage IV. In some embodiments, the pancreatic cancer is metastatic pancreatic cancer. In some embodiments, the pancreatic cancer is at stage MO. In some embodiments, the pancreatic cancer is at stage Ml. In some embodiments, the individual has distant metastasis. In some embodiments, the individual does not have distant metastasis. In some embodiments, the individual had pancreatic cancer of any one of stage I, II, III or IV at the time of diagnosis of pancreatic cancer. The treatment methods disclosed herein can lead to an increase in progression free survival and overall survival of the subject administered the combination therapy.

Some embodiments relate to a method of inhibiting proliferation of a pancreatic cancer cells, the method including contacting the pancreatic cancer cells with hydroxyureamethyl acylfulvene. In some embodiments, the contacting comprises administering an effective amount of hydroxyureamethyl acylfulvene to a subject having the pancreatic cancer cells.

Some embodiments relate to a method of inducing apoptosis in a pancreatic cancer cells, the method including contacting the pancreatic cancer cells with hydroxyureamethyl acylfulvene. In some embodiments, the contacting comprises administering an effective amount of hydroxyureamethyl acylfulvene to a subject having the pancreatic cancer cells. In some embodiments, the pancreatic tumor is a pancreatic adenocarcinoma.

The administration period can be a multi-week treatment cycle or as the tumor remains under control and the regimen is clinically tolerated. In some embodiments, a single dosage of hydroxyureamethyl acylfulvene or other therapeutic agent can be administered once a week, and preferably once on each of day 1 and day 8 of a three-week (21 day) treatment cycle. In some embodiments, a single dosage of hydroxyureamethyl acylfulvene or other therapeutic agent can be administered once a week, twice a week, three times per week, four times per week, five times per week, six times per week, or daily during a one-week, two-week, three-week, four-week, or five-week treatment cycle. The administration can be on the same or different day of each week in the treatment cycle.

Another embodiment includes a method for treating or determining the sensitivity of pancreatic cancer to a hydroxyureamethyl acylfulvene treatment by assessing the level of gene expression.

Another embodiment includes a method of treating pancreatic cancer in a subject, comprising: (a) obtaining or having obtained an expression level in a sample from a subject for a plurality of targets, wherein the plurality of targets one or more genes (e.g., PTGR1); (b) determining that the subject is sensitive to a treatment with a hydroxyureamethyl acylfulvene; and (c) administering a cancer treatment including a hydroxyureamethyl acylfulvene.

Another embodiment includes a method of treating pancreatic cancer in a subject in need thereof comprising administering to the subject a therapeutically effective amount of hydroxyureamethyl acylfulvene and also administering an additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the same. The additional therapy may be in the form of adjuvant or neoadjuvant therapy. In some embodiments, the additional therapy is the administration of an anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional treatment is applied before, during or/after the administration of hydroxyureamethyl acylfulvene.

Hydroxyureamethyl acylfulvene for use in accordance with the present invention can be mainly administered by parenteral administration, specifically including subcutaneous administration, intramuscular administration, intravenous administration, transcutaneous administration, intrathecal administration, epidural administration, intra joint administration and local administration, or may also be administered in various dosage forms, for example by oral administration if possible.

The injections for parenteral administration include for example sterile, aqueous or non-aqueous solutions, suspensions and emulsions. The aqueous solutions and suspensions include for example distilled water for injections and physiological saline. The non-aqueous solutions and suspensions include for example propylene glycol, polyethylene glycol, vegetable oils such as olive oil, alcohols such as ethanol, and Polysorbate 80 (under trade name). Such composition may contain auxiliary agents such as preservatives, moistening agents, emulsifying agents, dispersing agents, stabilizers (for example, lactose) and dissolution auxiliary agents (for example, meglumine). These are sterilized by filtering through bacteria-retaining filters, blending sterilizing agents, or irradiation. Alternatively, these may be produced once into a sterile solid composition and then dissolved or suspended in sterile water or sterile solvents for injections, prior to use.

In some embodiments, the pancreatic cancer can be selected from pancreatic adenocarcinoma, non-resectable pancreatic cancer, locally advanced pancreatic cancer, borderline resectable pancreatic cancer, locally advanced pancreatic ductal adenocarcinoma, borderline resectable pancreatic ductal adenocarcinoma, metastatic pancreatic cancer, chemotherapy-resistant pancreatic cancer, pancreatic ductal adenocarcinoma, squamous pancreatic cancer, pancreatic progenitor, immunogenic pancreatic cancer, aberrantly differentiated endocrine exocrine (ADEX) tumors, an exocrine pancreatic cancer, pancreatic intraepithelial neoplasia, intraductal papillary mucinous neoplasms, mucmous cystic neoplasms, mucmous pancreas cancer, adenosquamous carcinoma, signet ring cell carcinoma, hepatoid carcinoma, colloid carcinoma, undifferentiated carcinoma, undifferentiated carcinomas with osteoclast-like giant cells, a pancreatic cystic neoplasm, an islet cell tumor, a pancreas endocrine tumor, or a pancreatic neuroendocrine tumor.

The liquid composition for oral administration includes for example, pharmaceutically acceptable emulsions, liquids, suspensions, syrups and elixirs and contains inert diluents for general use, for example distilled water and ethanol. The composition may contain auxiliary agents such as moistening agents and suspending agents, sweetening agents, flavoring agents, aromatic agents and preservatives, other than the inert diluents.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the compound in the composition.

### Examples

### Cell Line Studies

Cell line studies showed LP-184 activity in a spectrum of cell line and patient-derived pancreatic cancer models that represent a variety of molecular and clinical subtypes of pancreatic cancer.

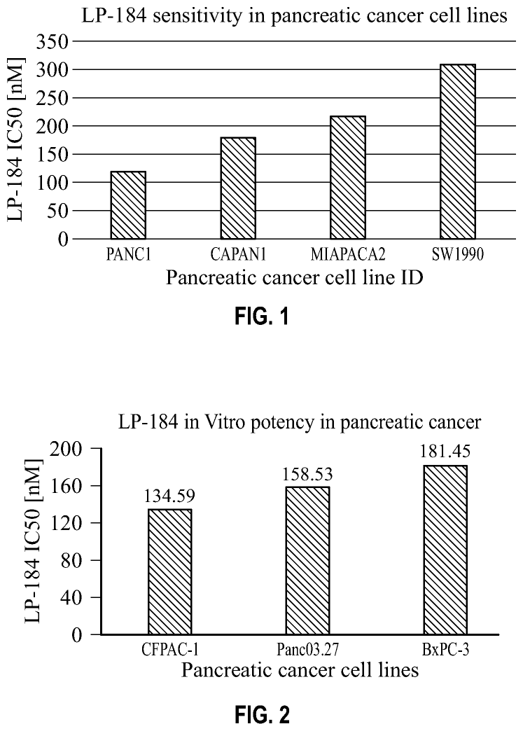

FIG. 1 and FIG. 2 show that LP-184 exhibited nanomolar potency in pancreatic cancer cell lines. Individual cell lines were treated with LP-184 for 72 hours and cell viability was assayed. IC50 values were generated from dose response curves plotted in GraphPad Prism. FIG. 1 shows the cytotoxicity of LP-184 in terms of IC50 values in a first study of 4 pancreatic cancer cell lines and the cytotoxicity ranged from about 110 to about 310 nM. FIG. 2 shows the cytotoxicity of LP-184 in terms of IC50 value in a second study of 4 pancreatic cancer cell lines and the cytotoxicity ranged from about 130 to about 180 nM.

### Ex Vivo Studies

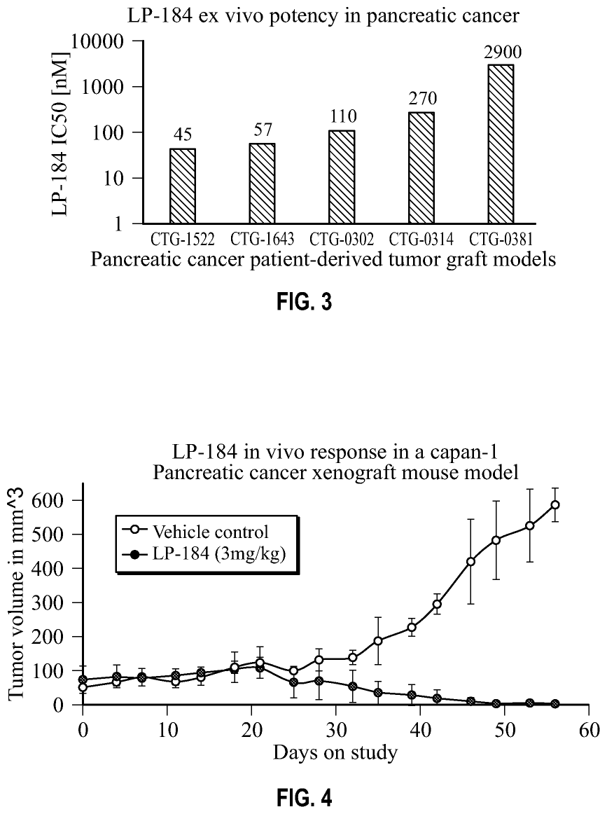

FIG. 3 shows IC50 values from tissue from prostate cancer tissue from models that were treated ex vivo with LP-184. Five low passage patient-derived tumor graft models were treated with LP-184. Patient-derived xenografts (PDX) are models of cancer where the tissue or cells from a patient's tumor are implanted into an immunodeficient or humanized mouse. Individual tumor models were treated with LP-184 for 120 hours and cell viability was assayed. IC50 values were generated from dose response curves plotted in GraphPad Prism.

### Animal Studies

All animal studies were carried out in compliance with National Institutes of Health Guidelines for animals. The doses were selected to be tolerated in xenografts. Capan-1 cell line derived xenograft tumors were implanted in SCID mice and treated either with (a) vehicle control of 95% saline/5% ethanol (N=3) or (b) 3 mg/kg LP-184 (N=5) administered by intraperitoneal injection over thrice weekly doses for 3 weeks. In other words, one group was treated with LP-184 and the other group was treated with the vehicle control.

FIG. 4 shows tumor volume growth in xenograft mouse models over 60 days. Complete tumor regression is observed in the LP-184 treated animals at the end of the study period or 60 days.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

1. A method of treating pancreatic cancer, in a subject in need thereof, the method comprising: administering to the subject a therapeutically effective amount of hydroxyureamethyl acylfulvene.

2. The method of claim 1, wherein the pancreatic cancer is endocrine.

3. The method of claim 1, wherein the pancreatic cancer is exocrine.

4. The method of claim 1, wherein the pancreatic cancer is pancreatic adenocarcinoma.

5. The method of claim 1, wherein the subject has a high level of PTGR1.

6. The method of claim 1, wherein the patient suffers from one or more of pancreatic adenocarcinoma, non-resectable pancreatic cancer, locally advanced pancreatic cancer, borderline resectable pancreatic cancer, locally advanced pancreatic ductal adenocarcinoma, borderline resectable pancreatic ductal adenocarcinoma, metastatic pancreatic cancer, chemotherapy-resistant pancreatic cancer, pancreatic ductaladenocarcinoma, squamous pancreatic cancer, pancreatic progenitor, immunogenic pancreatic cancer, aberrantly differentiated endocrine exocrine (ADEX) tumors, an exocrine pancreatic cancer, pancreatic intraepithelial neoplasia, intraductal papillary mucinous neoplasms, mucmous cystic neoplasms, mucmous pancreas cancer, adenosquamous carcinoma, signet ring cell carcinoma, hepatoid carcinoma, colloid carcinoma, undifferentiated carcinoma, undifferentiated carcinomas with osteoclast-like giant cells, a pancreatic cystic neoplasm, an islet cell tumor, a pancreas endocrine tumor, or a pancreatic neuroendocrine tumor.

7. The method of claim 6, wherein the pancreatic cancer is Stage I, II, III, or IV.

8. The method of claim 1, wherein the additional therapeutic agent is selected from the group consisting of temozolomide, bevacizumab, everolimus, carmustine, lomustine, procarbazine, vincristine, irinotecan, cisplatin, carboplatin, methotrexate, etoposide, vinblastine, bleomycin, actinomycin, cyclophosphamide, and ifosfamide.

9. The method of claim 1, wherein the additional therapeutic agent is selected from the group consisting of cisplatin, paclitaxel, and combination thereof.

10. The method of claim 1, further comprising subjecting the subject to radiation therapy.

11. The method of claim 1, wherein the cell is in a subject is an animal.

12. The method of claim 1, wherein the subject or mammal is a human.

13. The method of claim 1, wherein the method is for first-line treatment.

14. The method of claim 5, wherein the other therapies comprise surgery, chemotherapy or combinations thereof.

15. The method of claim 1, wherein the patient has undergone surgery.

16. The method of claim 1, wherein the patient has undergone radiation therapy.

17. The method of claim 1, wherein the patient has undergone treatment with chemotherapy.

18. A method of treating pancreatic cancer a subject, comprising: (a) obtaining or having obtained an expression level in a sample from a subject for a plurality of targets, wherein the plurality of targets comprises includes PTRG1; (b) determining that the subject is sensitive to a treatment with a hydroxyureamethyl acylfulvene; and (c) administering a cancer treatment including a hydroxyureamethyl acylfulvene.

19. The method of claim 18, wherein the patient has undergone radiation therapy.

20. The method of claim 18, wherein the additional therapeutic agent is selected from the group consisting of cisplatin, paclitaxel, and combination thereof.
